(19) United States
(12) Patent Application Publication
(10) Pub. No.: US 2017/0014456 A1
(43) Pub. Date: Jan. 19, 2017

(54) METHODS AND SYSTEMS FOR ENDOMETRIAL TREATMENT

(71) Applicant: Celmatix Inc., New York, NY (US)

(72) Inventor: Piraye Yurttas Beim, New York, NY (US)

(21) Appl. No.: 15/209,357

(22) Filed: Jul. 13, 2016

Related U.S. Application Data
(60) Provisional application No. 62/193,903, filed on Jul. 17, 2015.

(51) Int. Cl. A61K 35/48; C12N 5/071; C12N 15/90; A61K 9/00

(52) U.S. Cl. CPC A61K 35/48; A61K 9/0034; C12N 5/0682; C12N 15/907; C12N 2800/80; C12N 2506/243

(57) ABSTRACT

Methods of treating endometrial dysfunction are described including retrieval of endometrial cells including endometrial stem cells from a fertile donor and transplantation of endometrial cells or tissue into a host suffering from endometrial dysfunction. In certain embodiments, methods include genetic modification, tissue culturing, or cryopreservation of the endometrial cells prior to transplantation. In various aspects, endometrial cells are retrieved from an individual during peak fertility and banked for autologous transplantation at a later time.

### CROSS-REFERENCE TO RELATED APPLICATIONS

This application claims priority to, and the benefit of, U.S. Provisional Application No. 62/193,903, filed Jul. 17, 2015, which is incorporated by reference in its entirety.

### BACKGROUND

According to the Centers for Disease Control and Prevention, 6.7 million women (around 10.9%) in the United States between the ages of 15 and 44 suffer from impaired fecundity, or the ability to become pregnant and carry a baby to term. See Chandra A, Copen C E, Stephen E H. Infertility and impaired fecundity in the United States, 1982-2010: Data from the National Survey of Family Growth. National health statistics reports; no 67. Hyattsville, Md.: National Center for Health Statistics, 2013. For many women, infertility involves a problem with the implantation-attachment of the embryo to the uterus. Implantation depends both on the quality of the embryo and the ability of the endometrium (the inner lining of the uterus) to receive the embryo. The endometrium consists of two layers, the functional layer, closest to the uterine cavity, and the basal layer underneath the functional layer. The functional layer proliferates during each menstrual cycle before being completely shed during menstruation.

The readiness of the endometrium to accept an embryo is termed endometrial receptivity. Endometrial receptivity may be evaluated through ultrasound scan, endometrial biopsy, or through analysis of biochemical markers such as gene expression levels. See Bonilla-Musoles, et al., Endometrial receptivity: evaluation with ultrasound, Ultrasound Q. 2013 March; 29(1):3-20; Gómez, et al., Human Endometrial Transcriptomics: Implications for Embryonic Implantation, Cold Spring Harb Perspect Med. 2015 Mar. 27; Ruiz-Alonso, et al., The genomics of the human endometrium, Biochim Biophys Acta. 2012 December; 1822(12):1931-42; Garrido-Gómez, et al., Profiling the gene signature of endometrial receptivity: clinical results, Fertil Steril. 2013 Mar. 15; 99(4):1078-85; Elnashar, et al., Endometrial Receptivity, Middle East Fertility Society Journal, Vol. 9, No. 1, 2004, pp. 10-24; the contents of each of which are incorporated herein in their entirety.

After research focused on embryo retrieval, storage, and preparation, and continued improvement of the embryo transfer procedure, in vitro fertilization (IVF) success rates in strong candidates still hover around 50%. It has become apparent that endometrial function and receptivity are major limiting factors in the establishment of pregnancy. Endometrial receptivity can be negatively affected by a number of factors leading to endometrial dysfunction and repeated implantation failure (RIF). See, Strowitzki, et al., The human endometrium as a fertility-determining factor, Human Reproduction Update, Vol. 12, No. 5 pp. 617-630, 2006; Simon, et al., Assessment and treatment of repeated implantation failure (RIF), J Assist Reprod Genet (2012) 29:1227-1239; the contents of each of which are incorporated herein in their entirety. Known treatment methods include limited hormone, gene, and protein therapies, but they have failed to achieve widespread acceptance or success. Id.

### SUMMARY

The invention relates to methods and systems for assessing and treating endometrial dysfunction and endometrium-related infertility. Aspects of the invention include obtaining endometrial cells (e.g., endometrial stem cells (EnSC) or tissue from the basal or functional layer of the endometrium) from fertile donors and transplanting them into a host. Endometrial cells may be harvested from a woman at a time of heightened fertility (e.g., while the woman is near the beginning of her reproductive lifespan or in the months or years following childbirth) and then transplanted back into the donor's uterus at a later time, when the woman's endometrium is less proliferative or less receptive to implantation. Endometrial cells may be obtained from a donor by endometrial biopsy or isolated from menstrual fluid and may be cryopreserved for later use.

In certain embodiments, endometrial cells may be collected contemporaneously with embryo retrieval and frozen or cryopreserved along with embryos for use in future IVF procedures. Endometrial cells may be collected and stored for many reasons, including where a woman wishes to delay child bearing or where a woman is at risk of an endometrial or uterine disease or where a woman is to undergo a treatment which may negatively affect the endometrium. According to the invention, endometrial tissue or cells may be, for example, autologous, as described above, or may be allogenic and transplantation may be close in time to retrieval from a donor.

After retrieval, endometrial tissue or cells may be transplanted unchanged, subjected to genetic modification, or cultured into tissue and grafted. In certain autologous embodiments and especially where the donor/recipient suffers from endometrial dysfunction with a genetic link, the endometrial cells may be subjected to gene therapy using viral or liposome-mediated transfection or using a CRISPR/Cas system in order to restore or enhance a variety of endometrial cell functions.

Retrieval and transplantation may be timed according to the uterine cycle phase of the donor and host respectively. For example, endometrial tissue may be transplanted during the host's proliferative phase, within a few days of the end of menstruation in order to promote proliferation of the functional layer of the host's endometrium prior to embryo transfer.

Aspects of the invention include methods for treating endometrial dysfunction. Steps of the method include obtaining an endometrial stem cell from a donor and transplanting the endometrial stem cell into a host uterus. In certain embodiments, the donor may also be the host. Methods of the invention may include cryopreserving the endometrial stem cell.

The endometrial stem cell may be obtained within a 5 year period after the donor has given birth. The endometrial stem cell may be obtained by endometrial biopsy or isolated from menstrual fluid. In certain embodiments, the endometrial stem cell may be transplanted during the proliferative phase of the uterine cycle of the host. Methods of the invention may include injection of the endometrial stem cell into endometrium of the host uterus. Certain methods may include genetically modifying the endometrial stem cell before transplantation. The genetic modification may include using a clustered regularly interspaced short palindromic repeat (CRISPR) nuclease or may include augmenting expression of a protein linked to endometrial receptivity. Methods may include screening the donor for endometrial function or HLA-matching the donor and the host.

Certain aspects of the invention relate to a method for treating endometrial dysfunction including the steps of obtaining endometrial tissue from an individual, genetically modifying the endometrial tissue, and transplanting the endometrial tissue into the individual. The genetic modification may include using a clustered regularly interspaced short palindromic repeat (CRISPR) nuclease. In certain embodiments, the genetic modification comprises augmenting expression of a protein linked to endometrial receptivity.

In various aspects, methods of the invention include a method for treating endometrial dysfunction. Steps of the method include obtaining endometrial tissue from an individual, cryopreserving the endometrial tissue and transplanting the endometrial tissue into the individual. The endometrial tissue may be obtained within a 5 year period after the individual has given birth.

### BRIEF DESCRIPTION OF DRAWINGS

FIG. 1 provides a diagram of methods of the invention.

### DETAILED DESCRIPTION

Methods and systems of the invention generally relate to assessing and treating endometrial dysfunction and endometrium-related infertility. Endometrial tissue or stem cells may be obtained from a donor and transplanted into a host suffering from endometrial dysfunction prior to embryo transfer in IVF or natural conception in order to promote endometrial proliferation and receptivity. Endometrial cells may be obtained through biopsy or isolated from menstrual fluid and may be stored in cryopreservation alone or along with retrieved embryos for future transplantation. Endometrial cells may be modified after retrieval to correct genetic defects or to enhance selected endometrial functions before transplantation. In certain embodiments, endometrial cells may be collected from a woman and stored or modified before re-introduction into the same woman's uterus, enabling preservation of healthy, autologous endometrial cells to repopulate the endometrium later in life or after an event affecting endometrial function.

Methods of the invention involve obtaining endometrial tissue or endometrial stem cells (EnSCs), from a host. The cells may be collected in any clinically acceptable manner. A tissue is a mass of connected cells and/or extracellular matrix material derived from, for example, a human or other mammal and includes the connecting material and the liquid material in association with the cells and/or tissues. Endometrial cells may be obtained in the form of fine needle aspirate or biopsied tissue. Cells may be suspended or stored in media such as cell culture media. The endometrial tissue may be obtained over different time-points across the uterine cycle.

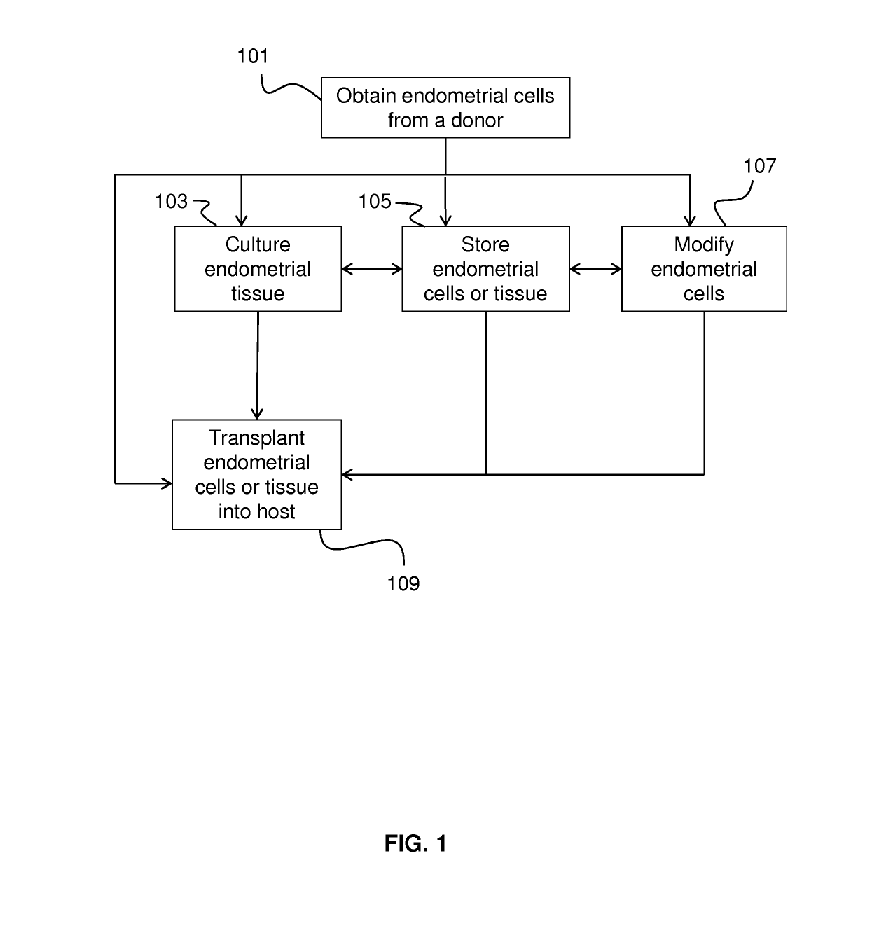

FIG. 1 illustrates an exemplary embodiment of a method of the invention. Endometrial cells are obtained from a donor 101 and may then be implanted into a host 109. The endometrial cells may optionally be modified 107 through, for example, genome editing, stored (e.g., cryopreserved), or cultured or engineered into a tissue 103 prior to transplantation 109. The optional steps may be performed in any combination and order, for example, cells may be modified or cultured into tissue before or after storage.

Prior to retrieval, a donor may be screened including blood, immunological, or nucleic acid analysis in order to determine health of the endometrial cells and compatibility with the host. For example, a donor and host may be HLA matched as in bone marrow or other stem cell transplantation techniques. See Petersdorf, HLA matching in allogeneic stem cell transplantation, Curr Opin Hematol. 2004 November; 11(6):386-91, incorporated herein in its entirety. In certain instances, HLA typing may be used to identify cells for transplantation which may be matched at certain markers to avoid rejection or donor vs. host disease but may be different at other markers relevant to implantation and repeated implantation failure. See Simon, et al., Assessment and treatment of repeated implantation failure (RIF), J Assist Reprod Genet (2012) 29:1227-1239, incorporated herein in its entirety. Significant percentages of host endometrial tissue have been found to carry HLA type of the donor after bone marrow transplant, indicating that endometrial cell transplantation may also alter the genetic make-up of the host endometrium. See, Taylor, Endometrial Cells Derived From Donor Stem Cells in Bone Marrow Transplant Recipients, JAMA. 2004 Jul. 7; 292(1):81-5.

### Retrieval

Endometrial stem cells may be isolated from a donor, for example, from hysterectomy, diagnostic curettage, menstrual blood, and first-trimester decidua. See Gargett, et al., Isolation and Culture of Epithelial Progenitors and Mesenchymal Stem Cells from Human Endometrium, BIOLOGY OF REPRODUCTION 80, 1136-1145 (2009); Xiaolong, et al., Endometrial regenerative cells: A novel stem cell population, J Transl Med. 2007, 5: 57; Lin, et al., Plasticity of human menstrual blood stem cells derived from the endometrium, J Zhejiang Univ Sci B. 2011 May; 12(5): 372-380; the contents of each of which are incorporated herein in their entirety.

In certain embodiments, endometrial stem cells may be isolated from menstrual fluid using, for example, the technique described in Xiaolong, et al., 2007. Endometrial stem cells may alternatively be isolated from endometrial tissue (see Gargett, et al., 2009) obtained, for example, through biopsy. In certain embodiments, whole endometrial tissue, such as basal or functional layer endometrial tissue, may be removed from the donor and transplanted in the host. See Grunberger, V, Successful endometrium transplantations, Geburtshilfe Frauenheilkd, 1969-May; vol 29 (issue 5): pp 478-81, incorporated herein in its entirety.

In certain embodiments, endometrial cells may be retrieved from the donor during a particular phase of the donor's uterine cycle. The uterine cycle that governs endometrial tissue has several different phases. The different phases are governed by hormone changes, and thus the phases vary from person to person. The uterine cycle begins with the menstrual or menstruation phase. The menstrual phase is the phase during which the endometrium is shed as menstrual flow. Menstrual fluid sheds out of the cervix and vagina. The first day of menstrual flow is defined as the first day of the menstrual cycle. The menstrual phase lasts about 3 to 7 days. During the menstrual phase, the pituitary glands begin to secrete follicle-stimulating hormone (FSH). The rise in FSH triggers the proliferation phase (Follicular). Endometrial cells may be retrieved, for example, during the proliferative phase when obtained by biopsy. Retrieval from menstrual fluid will obviously take place during the menstrual phase.

### Storage

In various embodiments, endometrial cells or tissues, whether modified or cultured as described below, or simply as retrieved from the donor, may be stored. Particularly in cases of autologous transplant, cells may be retrieved from an individual and banked for future use in case of future endometrial dysfunction or may be retrieved in anticipation of endometrial dysfunction related to, for example, age or treatment of a disease. Endometrial cells may be collected from a donor during a period of increased fertility for example, within 1, 2, 3, 4, 5, or 6 years of pregnancy or during the first several years of the woman's reproductive lifespan (beginning with puberty or regular ovulation). Endometrial cells may be stored using known methods such as cryopreservation as used in preservation of ovarian tissue, embryos and oocytes. See Oktay, et al., November 2008, Ovarian cryopreservation and transplantation for fertility preservation for medical indications: report of an ongoing experience, Fertil. Steril. 93 (3): 762-8; Edgar, et al., 2012 A critical appraisal of cryopreservation (slow cooling versus vitrification) of human oocytes and embryos, Human Reproduction Update 18 (5): 536; Kopeika, et al., 2014, The effect of cryopreservation on the genome of gametes and embryos: principles of cryobiology and critical appraisal of the evidence, Human Reproduction Update 21 (2): 209-227; the contents of each of which are incorporated herein in their entirety.

### Modification

After retrieval of endometrial stem cells or tissue from a host, and before implantation, the material may be genetically modified by a variety of known techniques to correct genetic defects or to enhance proliferation or receptivity of the endometrium post-transplant. For example endometrial cells may be modified to repair a mutation in a gene linked to an endometrial cancer such as PTEN. For additional genes linked to endometrial cancers, see Okuda, et al., Genetics of Endometrial Cancers, Obstetrics and Gynecology International, vol. 2010, Article ID 984013, 8 pages, 2010, incorporated herein in its entirety. Endometrial cells may be modified to change expression of endometriosis linked genes such as those described in Kao, et al., Expression profiling of endometrium from women with endometriosis reveals candidate genes for disease-based implantation failure and infertility, Endocrinology. 2003 July; 144(7):2870-81, incorporated herein in its entirety. The following is a list of genes whose expression levels correlate significantly with endometriosis: CCL3L1, CCL3, FAM180A, THBS2, PDGFRL, FN1, CLE11A, CCNA2, KIF20A, BUB1B, HSD17B6, HSD11B1, C7, C3, CXCL2, CXCL12, CXCL13, PDGFC, CXCL14, ACTA2, TAGLN, and SORBS1. De-regulated genes associated with endometriosis during the proliferative phase include CCNA2, KIF20A, BUB1B. Up-regulated genes associated with endometriosis during the proliferative phase include HSD17B6, HSD11B1, C7, C3, CXCL2, CXCL12, CXCL14. De-regulated genes associated with endometriosis during the early secretory phase include CXCL13. Up-regulated genes associated with endometriosis during the early secretory phase include CCNA2, KIF20A, BUB1B. Up-regulated genes associated with the endometriosis during the mid- to late secretory phase include ACTA2, TAGLN, and SORBS1. Phase-specific genes associated with endometriosis are also described in: Hawkins, Shannon M., et al. “Functional microRNA involved in endometriosis.” Molecular endocrinology 25.5 (2011): 821-832; Sha, G., et al. “Differentially expressed genes in human endometrial endothelial cells derived from eutopic endometrium of patients with endometriosis compared with those from patients without endometriosis.” Human reproduction 22.12 (2007): 3159-3169; Burney, Richard O., et al. “Gene expression analysis of endometrium reveals progesterone resistance and candidate susceptibility genes in women with endometriosis.” Endocrinology 148.8 (2007): 3814-3826; Crispi, Stefania, et al. “Transcriptional profiling of endometriosis tissues identifies genes related to organogenesis defects.” Journal of cellular physiology 228.9 (2013): 1927-1934; Eyster, Kathleen M., et al. “Whole genome deoxyribonucleic acid microarray analysis of gene expression in ectopic versus eutopic endometrium.” Fertility and sterility 88.6 (2007): 1505-1533; Hever, Aniko, et al. “Human endometriosis is associated with plasma cells and overexpression of B lymphocyte stimulator.” Proceedings of the National Academy of Sciences 104.30 (2007): 12451-12456; Hull, M. Louise, et al. “Endometrial-peritoneal interactions during endometriotic lesion establishment.” The American journal of pathology 173.3 (2008): 700-715; Talbi, S., et al. “Molecular phenotyping of human endometrium distinguishes menstrual cycle phases and underlying biological processes in normo-ovulatory women.” Endocrinology 147.3 (2006): 1097-1121.

In certain embodiments endometrial cells may be genetically modified to alter expression of proteins such as HOXA10, LIF, IL-6, MFAP5, ANGPTL1, EG-VEGF, NLF2. See Strowitzki, et al, 2006; Martin, et al., Human endometrial receptivity: gene regulation, J Reprod Immunol. 2002 May-June; 55(1-2):131-9; Riesewijk, et al., Gene expression profiling of human endometrial receptivity on days LH+2 versus LH+7 by microarray technology, Molecular Human Reproduction Vol. 9, No. 5 pp. 253-264, 2003; Haozi, et al., Gene expression profile of human endometrial receptivity: comparison between natural and stimulated cycles for the same patients, Hum Reprod. 2009 June; 24(6): 1436-1445; the contents of each of which are incorporated herein in their entirety.

In certain embodiments, nucleic acid may be extracted from endometrial tissue or cells retrieved from a donor by any of the means described above for sequencing or other analysis to determine health of the cells or to search for genetic defects or mutations linked to endometrial dysfunction. In various embodiments, a small amount of endometrial tissue or cells may be retrieved and analyzed as part of a screening procedure before retrieval of additional cells or tissue for transplantation purposes.

Nucleic acid may be extracted from the endometrial sample according to methods known in the art. See for example, Maniatis, et al., Molecular Cloning: A Laboratory Manual, Cold Spring Harbor, N.Y., pp. 280-281, 1982, the contents of which are incorporated by reference herein in their entirety. In certain embodiments, a genomic sample is collected from a subject (e.g., host, donor, or potential donor) followed by enrichment for genetic regions or genetic fragments of interest, for example by hybridization to a nucleotide array comprising endometrial-related genes or gene fragments of interest. The sample may be enriched for genes of interest (e.g., endometrial-associated genes) using methods known in the art, such as hybrid capture. See for examples, Lapidus (U.S. Pat. No. 7,666,593), the content of which is incorporated by reference herein in its entirety.

Nucleic acids extracted from endometrial samples may be analyzed through a number of means including sequencing or expression or transcript analysis.

Extracted nucleic acid may be amplified prior to analysis. Amplification methods include, for example, amplification of a single target nucleic acid and multiplex amplification (amplification of multiple target nucleic acids in parallel). Amplification refers to production of additional copies of a nucleic acid sequence and is generally conducted using polymerase chain reaction (PCR) or other technologies well-known in the art (e.g., Dieffenbach and Dveksler, PCR Primer, a Laboratory Manual, 1995, Cold Spring Harbor Press, Plainview, N.Y.). The amplification reaction may be any amplification reaction known in the art that amplifies nucleic acid molecules, such as polymerase chain reaction, nested polymerase chain reaction, polymerase chain reaction-single strand conformation polymorphism, ligase chain reaction (Barany, F. Genome research, 1:5-16 (1991); Barany, F., PNAS, 88:189-193 (1991); U.S. Pat. No. 5,869,252; and U.S. Pat. No. 6,100,099), strand displacement amplification and restriction fragment length polymorphism, transcription based amplification system, rolling circle amplification, and hyper-branched rolling circle amplification. Further examples of amplification techniques that can be used include, without limitation, quantitative PCR, quantitative fluorescent PCR (QF-PCR), multiplex fluorescent PCR (MF-PCR), real time PCR (RTPCR), single cell PCR, restriction fragment length polymorphism (PCR-RFLP), RT-PCR-RFLP, hot start PCR, in situ polonony PCR, in situ rolling circle amplification (RCA), bridge PCR, picotiter PCR, and emulsion PCR. Other suitable amplification methods include transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938.

In various aspects, methods of the invention relate to sequencing of nucleic acid samples isolated from somatic cells of the individual or the sequencing of circulating cell-free nucleic acid. Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, translocation through a nanopore or nanochannel, digestion or polymerization of DNA combined with detection of nucleotides in a nanopore or nanochannel, optical detection of nucleotides in strands localized with a nanopore or nanochannel, and SOLiD sequencing. Separated molecules may be sequenced by sequential or single extension reactions using polymerases or ligases as well as by single or sequential differential hybridizations with libraries of probes.

In some embodiments, a sequencing technique (e.g., a next-generation sequencing technique) is used to sequence part of one or more captured targets (e.g., or amplicons thereof) and the sequences are used to count the number of different barcodes that are present. Accordingly, in some embodiments, aspects of the invention relate to a highly-multiplexed qPCR reaction.

A sequencing technique that can be used includes, for example, Illumina sequencing. Illumina sequencing is based on the amplification of DNA on a solid surface using fold-back PCR and anchored primers. DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pat. No. 7,960,120; U.S. Pat. No. 7,835,871; U.S. Pat. No. 7,232,656; U.S. Pat. No. 7,598,035; U.S. Pat. No. 6,911,345; U.S. Pat. No. 6,833,246; U.S. Pat. No. 6,828,100; U.S. Pat. No. 6,306,597; U.S. Pat. No. 6,210,891; U.S. Pub. 2011/0009278; U.S. Pub. 2007/0114362; U.S. Pub. 2006/0292611; and U.S. Pub. 2006/0024681, each of which is incorporated by reference in their entirety.

Sequencing generates a plurality of reads. Reads generally include sequences of nucleotide data less than about 150 bases in length, or less than about 90 bases in length. In certain embodiments, reads are between about 80 and about 90 bases, e.g., about 85 bases in length. In some embodiments, these are very short reads, i.e., less than about 50 or about 30 bases in length.

A sequencing technique that can be used in the methods of the provided invention includes, for example, 454 sequencing (454 Life Sciences, a Roche company, Branford, Conn.) (Margulies, M et al., Nature, 437:376-380 (2005); U.S. Pat. No. 5,583,024; U.S. Pat. No. 5,674,713; and U.S. Pat. No. 5,700,673). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used in the methods of the provided invention is SOLiD technology by Applied Biosystems from Life Technologies Corporation (Carlsbad, Calif.). In SOLiD sequencing, DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

Another example of a DNA sequencing technique that can be used in the methods of the provided invention is Ion Torrent sequencing, described, for example, in U.S. Pubs. 2009/0026082, 2009/0127589, 2010/0035252, 2010/0137143, 2010/0188073, 2010/0197507, 2010/0282617, 2010/0300559, 2010/0300895, 2010/0301398, and 2010/0304982, the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and are attached at a resolution such that the fragments are individually resolvable. Addition of one or more nucleotides releases a proton (H.sup.+), which signal is detected and recorded in a sequencing instrument. The signal strength is proportional to the number of nucleotides incorporated.

Another example of a sequencing technology that can be used in the methods of the provided invention is Illumina sequencing. Illumina sequencing is based on the amplification of DNA on a solid surface using fold-back PCR and anchored primers. DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pub. 2011/0009278, U.S. Pub. 2007/0114362, U.S. Pub. 2006/0024681, U.S. Pub. 2006/0292611, U.S. Pat. No. 7,960,120, U.S. Pat. No. 7,835,871, U.S. Pat. No. 7,232,656, U.S. Pat. No. 7,598,035, U.S. Pat. No. 6,306,597, U.S. Pat. No. 6,210,891, U.S. Pat. No. 6,828,100, U.S. Pat. No. 6,833,246, and U.S. Pat. No. 6,911,345, each of which are herein incorporated by reference in their entirety.

Another example of a sequencing technology that can be used in the methods of the invention includes the single molecule, real-time (SMRT) technology of Pacific Biosciences (Menlo Park, Calif.). In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used in the methods of the provided invention is nanopore sequencing (Soni, G. V., and Meller, A., Clin Chem 53: 1996-2001 (2007)). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used in the methods of the provided invention involves using a chemical-sensitive field effect transistor (chemFET) array to sequence DNA (for example, as described in U.S. Pub. 2009/0026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used in the methods of the provided invention involves using an electron microscope (Moudrianakis E. N. and Beer M., PNAS, 53:564-71(1965)). In one example of the technique, individual DNA molecules are labeled using metallic labels that are distinguishable using an electron microscope. These molecules are then stretched on a flat surface and imaged using an electron microscope to measure sequences.

Another example of a sequencing technique that can be used in the methods of the provided invention involves Fast Aneuploidy Screening Test-Sequencing System (FAST-SeqS), as described in PCT application PCT/US2013/033451, which is incorporated by reference. See also Kinde et al., “FAST-SeqS: A Simple and Efficient Method for the Detection of Aneuploidy by Massively Parallel Sequencing,” DOI: 10.1371/journal.pone.0041162, which is incorporated by reference. FAST-SeqS uses specific primers, specifically, a single pair of primers that anneal to a subset of sequences dispersed throughout the genome. The regions are selected due to similarity so that they could be amplified with a single pair of primers, but sufficiently unique to allow most of the amplified loci to be distinguished. FAST-SeqS yielded sequences align to a smaller number of positions, as opposed to traditional whole genome amplification libraries in which each tag must be independently aligned.

Sequence assembly can be accomplished by methods known in the art including reference-based assemblies, de novo assemblies, assembly by alignment, or combination methods. In some embodiments, sequence assembly uses the low coverage sequence assembly software (LOCAS) tool described by Klein, et al., in LOCAS-A low coverage sequence assembly tool for re-sequencing projects, PLoS One 6(8) article 23455 (2011), the contents of which are hereby incorporated by reference in their entirety. Sequence assembly is described in U.S. Pat. No. 8,165,821; U.S. Pat. No. 7,809,509; U.S. Pat. No. 6,223,128; U.S. Pub. 2011/0257889; and U.S. Pub. 2009/0318310, the contents of each of which are hereby incorporated by reference in their entirety.

RNA may be isolated from eukaryotic cells by procedures that involve lysis of the cells and denaturation of the proteins contained therein. Tissue of interest includes gametic cells, gonadal tissue, endometrial tissue, fertilized embryos, and placenta. RNA may be isolated from fluids of interest by procedures that involve denaturation of the proteins contained therein. Fluids of interest include blood, menstrual fluid, mammary fluid, follicular fluid of the ovary, peritoneal fluid, or culture medium. Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., Biochemistry 18:5294-5299 (1979)). Poly(A)+RNA is selected by selection with oligo-dT cellulose (see Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol. If desired, RNase inhibitors may be added to the lysis buffer. Likewise, for certain cell types, it may be desirable to add a protein denaturation/digestion step to the protocol.

For many applications, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or SEPHADEX (see Ausubel et al., CURRENT PROTOCOLS IN MOLECULAR BIOLOGY, vol. 2, Current Protocols Publishing, New York (1994). Once bound, poly(A)+mRNA is eluted from the affinity column using 2 mM EDTA/0.1% SDS.

According to certain embodiments, expression levels of a host or potential donor are compared to reference data specific to phase in the uterine cycle. The reference data may be phase-specific signatures of normal tissue in fertile women. Differences in the signature, especially in the host/donor in autologous transplantation, may be analyzed to characterize regulation patterns associated with their endometrial dysfunction and to target specific genes for modification before transplantation.

In certain aspects, the invention involves assessing transcripts present in a biological sample such as endometrial cells from a host or potential donor. Such methods may involve preparing amplified cDNA from total RNA. cDNA is prepared and indiscriminately amplified without diluting the isolated RNA sample or distributing the mixture of genetic material in the isolated RNA into discrete reaction samples. Preferably, amplification is initiated at the 3′ end as well as randomly throughout the whole transcriptome in the sample to allow for amplification of both mRNA and non-polyadenylated transcripts. The double-stranded cDNA amplification products are thus optimized for the generation of sequencing libraries for Next Generation Sequencing platforms. Suitable kits for amplifying cDNA in accordance with the methods of the invention include, for example, the Ovation® RNA-Seq System.

Methods of the invention may also involve sequencing the amplified cDNA. Any known sequencing method can be used to sequence the amplified cDNA mixture including single molecule sequencing methods. Amplified cDNA may be sequenced by whole transcriptome shotgun sequencing (also referred to herein as (“RNA-Seq”). Whole transcriptome shotgun sequencing (RNA-Seq) can be accomplished using a variety of next-generation sequencing platforms such as the Illumina Genome Analyzer platform, ABI Solid Sequencing platform, or Life Science's 454 Sequencing platform.

Differential transcript levels within host or potential donor endometrial samples can also be analyzed using via microarray techniques. The amplified cDNA can be used to probe a microarray containing gene transcripts associated with one or conditions or diseases, such as any prenatal condition, or any type of cancer, inflammatory, or autoimmune disease.

In certain aspects, the invention provides a microarray including a plurality of oligonucleotides attached to a substrate at discrete addressable positions, in which at least one of the oligonucleotides hybridizes to a portion of a gene from Table 1 that includes an infertility-associated mutation. In certain embodiments

Methods of constructing microarrays are known in the art. See for example Yeatman et al. (U.S. patent application number 2006/0195269), the content of which is hereby incorporated by reference in its entirety.

Microarrays are prepared by selecting probes that include a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

The probe or probes used in the methods of the invention are preferably immobilized to a solid support which may be either porous or non-porous. For example, the probes of the invention may be polynucleotide sequences which are attached to a nitrocellulose or nylon membrane or filter covalently at either the 3′ or the 5′ end of the polynucleotide. Such hybridization probes are well known in the art (see, e.g., Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, the solid support or surface may be a glass or plastic surface. In certain embodiments, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

A microarray may comprise a support or surface with an ordered array of binding (e.g., hybridization) sites or “probes” each representing one of the genes described herein, particularly the genes described in Table 1. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). In certain embodiments, each probe is covalently attached to the solid support at a single site.

Microarrays can be made in a number of ways, of which several are described below. However produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. The microarrays are preferably small, e.g., between 1 cm² and 25 cm², between 12 cm² and 13 cm², or 3 cm². However, larger arrays are also contemplated and may be preferable, e.g., for use in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

The microarrays of the present invention include one or more test probes, each of which has a polynucleotide sequence that is complementary to a subsequence of RNA or DNA to be detected. Preferably, the position of each probe on the solid surface is known. Indeed, the microarrays are preferably positionally addressable arrays. Specifically, each probe of the array is preferably located at a known, predetermined position on the solid support such that the identity (i.e., the sequence) of each probe can be determined from its position on the array (i.e., on the support or surface).

According to the invention, the microarray is an array (i.e., a matrix) in which each position represents one of the biomarkers described herein. For example, each position can contain a DNA or DNA analogue based on genomic DNA to which a particular RNA or cDNA transcribed from that genetic marker can specifically hybridize. The DNA or DNA analogue can be, e.g., a synthetic oligomer or a gene fragment. In one embodiment, probes representing each of the markers are present on the array. In certain embodiments, the array comprises probes for genes known to be associated with endometriosis. In addition, the array probes may be specific to genes known to be associated with endometriosis at a certain phase of the uterine cycle.

As noted above, the probe to which a particular polynucleotide molecule specifically hybridizes according to the invention contains a complementary genomic polynucleotide sequence. The probes of the microarray preferably consist of nucleotide sequences of no more than 1,000 nucleotides. In some embodiments, the probes of the array consist of nucleotide sequences of 10 to 1,000 nucleotides. In various embodiments, the nucleotide sequences of the probes are in the range of 10-200 nucleotides in length and are genomic sequences of a species of organism, such that a plurality of different probes is present, with sequences complementary and thus capable of hybridizing to the genome of such a species of organism, sequentially tiled across all or a portion of such genome. In other specific embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, and most preferably are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates.

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR PROTOCOLS: A GUIDE TO METHODS AND APPLICATIONS, Academic Press Inc., San Diego, Calif. (1990). It will be apparent to one skilled in the art that controlled robotic systems are useful for isolating and amplifying nucleic acids.

An alternative means for generating the polynucleotide probes of the microarray is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568 (1993); U.S. Pat. No. 5,539,083).

Probes are preferably selected using an algorithm that takes into account binding energies, base composition, sequence complexity, cross-hybridization binding energies, and secondary structure. See Friend et al., International Patent Publication WO 01/05935, published Jan. 25, 2001; Hughes et al., Nat. Biotech. 19:342-7 (2001).

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes are attached to a solid support or surface, which may be made, e.g., from glass, plastic (e.g., polypropylene, nylon), polyacrylamide, nitrocellulose, gel, or other porous or nonporous material. One method for attaching the nucleic acids to a surface is by printing on glass plates, as is described generally by Schena et al, Science 270:467-470 (1995). This method is especially useful for preparing microarrays of cDNA (See also, DeRisi et al, Nature Genetics 14:457-460 (1996); Shalon et al., Genome Res. 6:639-645 (1996); and Schena et al., Proc. Natl. Acad. Sci. U.S.A. 93:10539-11286 (1995)).

A second method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids. Res. 20:1679-1684), may also be used. In principle, and as noted supra, any type of array, for example, dot blots on a nylon hybridization membrane (see Sambrook et al., MOLECULAR CLONING—A LABORATORY MANUAL (2ND ED.), Vols. 1-3, Cold Spring Harbor Laboratory, Cold Spring Harbor, N.Y. (1989)) could be used. However, as will be recognized by those skilled in the art, very small arrays will frequently be preferred because hybridization volumes will be smaller.

In one embodiment, the arrays of the present invention are prepared by synthesizing polynucleotide probes on a support. In such an embodiment, polynucleotide probes are attached to the support covalently at either the 3′ or the 5′ end of the polynucleotide.

In certain embodiments, microarrays of the invention are manufactured by means of an ink jet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells, which define the locations of the array elements (i.e., the different probes). Microarrays manufactured by this ink-jet method are typically of high density, preferably having a density of at least about 2,500 different probes per 1 cm.sup.2. The polynucleotide probes are attached to the support covalently at either the 3′ or the 5′ end of the polynucleotide.

The polynucleotide molecules which may be analyzed by the present invention are DNA, RNA, or protein. The target polynucleotides are detectably labeled at one or more nucleotides. Any method known in the art may be used to detectably label the target polynucleotides. Preferably, this labeling incorporates the label uniformly along the length of the DNA or RNA, and more preferably, the labeling is carried out at a high degree of efficiency.

In certain embodiments, the detectable label is a luminescent label. For example, fluorescent labels, bioluminescent labels, chemiluminescent labels, and colorimetric labels may be used in the present invention. In some embodiments, the label is a fluorescent label, such as a fluorescein, a phosphor, a rhodamine, or a polymethine dye derivative. Examples of commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.). In another embodiment, the detectable label is a radiolabeled nucleotide.

In a further embodiment, target polynucleotide molecules from a patient sample are labeled differentially from target polynucleotide molecules of a reference sample. The reference can comprise target polynucleotide molecules from normal tissue samples.

Nucleic acid hybridization and wash conditions are chosen so that the target polynucleotide molecules specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook et al., MOLECULAR CLONING—A LABORATORY MANUAL (2ND ED.), Vols. 1-3, Cold Spring Harbor Laboratory, Cold Spring Harbor, N.Y. (1989), and in Ausubel et al., CURRENT PROTOCOLS IN MOLECULAR BIOLOGY, vol. 2, Current Protocols Publishing, New York (1994). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Schena et al., Proc. Natl. Acad. Sci. U.S.A. 93:10614 (1993)). Useful hybridization conditions are also provided in, e.g., Tijessen, 1993, HYBRIDIZATION WITH NUCLEIC ACID PROBES, Elsevier Science Publishers B.V.; and Kricka, 1992, NONISOTOPIC DNA PROBE TECHNIQUES, Academic Press, San Diego, Calif.

Hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

When fluorescently labeled genes or gene products are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Research 6:639-645, which is incorporated by reference in its entirety for all purposes). In certain embodiments, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14:1681-1684 (1996) (the contents of which are incorporated herein in their entirety), may be used to monitor mRNA abundance levels at a large number of sites simultaneously.

After a desired genetic modification is determined, a variety of genetic or genome editing techniques may be used to accomplish the desired genetic modification. Genetic or genome editing techniques include zinc-finger domain methods, viral transcription activator-like effector nucleases (TALENs), liposome-mediated gene transfer, or clustered regularly interspaced short palindromic repeat (CRISPR) nucleases.

TALENs uses a nonspecific DNA-cleaving nuclease fused to a DNA-binding domain that can target essentially any sequence. For TALEN technology, target sites are identified and expression vectors are made. See Liu et al, 2012, Efficient and specific modifications of the Drosophila genome by means of an easy TALEN strategy, J. Genet. Genomics 39:209-215, the contents of which are incorporated herein in their entirety. A commercially available kit may be use such as the mMESSAGE mMACHINE SP6 transcription kit from Life Technologies (Carlsbad, Calif.). See Joung & Sander, 2013, TALENs: a widely applicable technology for targeted genome editing, Nat Rev Mol Cell Bio 14:49-55, the contents of which are incorporated herein in their entirety.

CRISPR methodologies employ a nuclease, CRISPR-associated (Cas9), together with small RNAs that act as guides (gRNAs) to cleave DNA in a sequence-specific manner upstream of a protospacer adjacent motif (PAM). Cas9 and guide RNA (gRNA) may be synthesized by known methods. CRISPR may use separate guide RNAs known as the crRNA and tracrRNA. These two separate RNAs have been combined into a single RNA to enable site-specific mammalian genome cutting through the design of a short guide RNA. Cas9/guide-RNA (gRNA) uses a non-specific DNA cleavage protein Cas9, and an RNA oligo to hybridize to target and recruit the Cas9/gRNA complex. See Chang et al., 2013, Genome editing with RNA-guided Cas9 nuclease in zebrafish embryos, Cell Res 23:465-472; Hwang et al., 2013, Efficient genome editing in zebrafish using a CRISPR-Cas system, Nat. Biotechnol 31:227-229; Xiao et al., 2013, Chromosomal deletions and inversions mediated by TALENS and CRISPR/Cas in zebrafish, Nucl Acids Res 1-11; the contents of each of which are incorporated herein in their entirety.

In certain embodiments, genome editing is performed using zinc finger nuclease-mediated process or liposome mediated gene transfer. See U.S. Pub. 2011/0023144 to Weinstein; Mannino, et al., Liposome mediated gene transfer, BioTechniques 1988, 6(7):682-690; the contents of each of which are incorporated herein in their entirety.

In various embodiments endometrial tissue may be prepared from endometrial cells through tissue culture techniques such as those described in Gargett, et al., 2009 or Zhong, et al., Feasibility investigation of allogeneic endometrial regenerative cells, Journal of Translational Medicine 2009, 7:15, incorporated herein in its entirety. In certain embodiments, tissue engineering techniques may be used to create endometrial grafts for transplantation from donor endometrial cells. See, Chen, et al., Stem Cells for Skin Tissue Engineering and Wound Healing, Crit Rev Biomed Eng. 2009; 37(4-5): 399-421, incorporated herein in its entirety.

In certain embodiments, endometrial cells or tissue obtained from a donor may be exposed to a therapeutic compound before being transplanted into a host uterus. Examples of therapeutic compounds may include, for example, seminal plasma; antibiotics; hormones such as FSH, estrogen, luteinizing hormone (LH), progesterone, or chorionic gonadotropin; enzymes; and cytokines or growth factors such as epidermal growth factor, fibroblast growth factor, transforming growth factor alpha (TGF)-α, platelet-derived growth factor (PDGF), transforming growth factor β1 (TGF-β1), interleukin 8 (IL-8), IL-1, IL-11, IL-15, migration inhibitory factor (MIF) and vascular endothelial growth factor (VEGF).

### Transplantation

According to systems and methods of the invention, endometrial stem cells may be introduced into the uterus of a host or recipient according to a variety of techniques such as trasmyometrial injection into the sub-endometrial zone or by introduction, with or without curettage, by catheter into the uterine cavity near the fundus. See Singh, et al., Autologous stem cell transplantation in refractory Asherman's syndrome: A novel cell based therapy; J Hum Reprod Sci 2014 April-June; 7(2): 93-98; Nagori, et al., Endometrial regeneration using autologous adult stem cells followed by conception by in vitro fertilization in a patient of severe Asherman's syndrome, J Hum Reprod Sci. 2011 January; 4(1):43-8; the contents of each of which are incorporated herein in their entirety. In certain embodiments endometrial tissue may be transplanted or grafted into the uterus of the host.

Transplantation may be accompanied by supplemental estrogen to signal endometrial tissue to thicken. In certain embodiments, the trigger for transplantation of endometrial cells may be the end of menstruation or measured changes in hormone levels (such as FSH, estrogen, or LH) in the blood or urine. Transplantation may be followed by IVF embryo transfer or other assistive reproductive technique within the same uterine cycle of the host.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore

What is claimed is:

1. A method for enhancing endometrial function and fertility, the method comprising:
obtaining an endometrial stem cell from a donor; and
transplanting the endometrial stem cell into a host uterus.

2. The method of claim 1 wherein the donor is also the host.

3. The method of claim 1 further comprising cryopreserving the endometrial stem cell.

4. The method of claim 1 wherein the endometrial stem cell is obtained within a 5 year period after the donor has given birth.

5. The method of claim 1 wherein the endometrial stem cell is obtained by endometrial biopsy.

6. The method of claim 1 wherein the endometrial stem cell is obtained by isolation from menstrual fluid.

7. The method of claim 1 wherein the endometrial stem cell is transplanted during the proliferative phase of the uterine cycle of the host.

9. The method of claim 1, wherein the transplantation step further comprises:
injection of the endometrial stem cell into endometrium of the host uterus.

10. The method of claim 1, further comprising genetically modifying the endometrial stem cell before transplantation.

11. The method of claim 10, wherein the genetic modification comprises using a clustered regularly interspaced short palindromic repeat (CRISPR) nuclease.

12. The method of claim 10, wherein the genetic modification comprises augmenting expression of a protein linked to endometrial receptivity.

13. The method of claim 1, further comprising screening the donor for endometrial function.

14. The method of claim 1, further comprising HLA-matching the donor and the host.

15. The method of claim 1, further comprising exposing the endometrial stem cell to a therapeutic compound.

16. The method of claim 1, further comprising culturing an endometrial tissue from the endometrial stem cell prior to the transplantation step.

17. The method of claim 1, further comprising differentiating the endometrial stem cell prior to the transplantation step.

18. The method of claim 17, further comprising exposing the differentiated cell to a therapeutic compound.

19. A method for enhancing endometrial function and fertility, the method comprising:
obtaining endometrial tissue from an individual;
genetically modifying the endometrial tissue; and
transplanting the endometrial tissue into the individual.

20. The method of claim 19, wherein the genetic modification comprises using a clustered regularly interspaced short palindromic repeat (CRISPR) nuclease.

21. The method of claim 19, wherein the genetic modification comprises augmenting expression of a protein linked to endometrial receptivity.

22. The method of claim 19, further comprising exposing the endometrial tissue to a therapeutic compound.

23. A method for enhancing endometrial function and fertility, the method comprising:
obtaining endometrial tissue from an individual;
cryopreserving the endometrial tissue; and
transplanting the endometrial tissue into the individual.

24. The method of claim 23, wherein the endometrial tissue is obtained within a 5 year period after the individual has given birth.

25. The method of claim 23, further comprising exposing the endometrial tissue to a therapeutic compound.
